(19) United States
(12) Patent Application Publication
(10) Pub. No.: US 2015/0312212 A1
(43) Pub. Date: Oct. 29, 2015

(54) HOLISTIC EMBODIMENT OF DNA AND IPV6

(71) Applicants: David Holmes, Brockville (CA); Michael Ian Ballerscheff, Brockville (CA)

(72) Inventors: David Holmes, Brockville (CA); Michael Ian Ballerscheff, Brockville (CA)

(21) Appl. No.: 14/696,340

(22) Filed: Apr. 24, 2015

Related U.S. Application Data
(60) Provisional application No. 61/983,570, filed on Apr. 24, 2014.

(51) Int. Cl. H04L 29/12; C12Q 1/68

(52) U.S. Cl. CPC H04L 61/2557; H04L 61/2521; C12Q 1/6876; C12Q 2600/158

(57) ABSTRACT

A method and apparatus for utilizing the specific genetic code of an organism, as defined by the organism's DNA, to define the unique and specific IPv6 address of the organism is provided. The IPv6 address may be within existing digital network infrastructures and may be decoded to define the organism's DNA sequence, which uniquely and accurately defines the organism's digital presence within the network. The described method and apparatus utilizes an algorithm or conversion formula to convert any DNA sequence into a digital IP address via an Alpha-binary-hexagonal converter. The same method may also define an organism's DNA by virtue of converting the IPv6 address into a DNA sequence, thereby allowing for DNA to be generated from an IPv6 address.

### CROSS REFERENCE TO RELATED APPLICATION

The present application claims the benefits of and priority from U.S. Provisional Application Ser. No. 61/983,570 filed Apr. 24, 2014, and entitled “Holistic Embodiment of DNA and IPV6,” the entire disclosure of which is hereby incorporated by reference in its entirety for all that it teaches and for all purposes.

### FIELD OF THE INVENTION

The present invention relates generally to a process and apparatus for converting a DNA sequence into an Internet Protocol address. More specifically, the IPv6 address associated with the DNA sequence may define the unique and specific IPv6 address of the organism. Likewise, the IPv6 address may be used to define a unique DNA sequence.

### REFERENCE TO SEQUENCE LISTING

This application contains a Sequence Listing submitted as an electronic text file named “7819-1_Sequence_Listing_ST25.txt,” having a size of 1 KB and created on May 8, 2015. The information contained in this electronic file is hereby incorporated by reference in its entirety pursuant to 37 CFR §1.52(e)(5).

### BACKGROUND

According to Wikipedia, the Internet of Things (IoT) is the network of physical objects or “things” embedded with electronics, software, sensors and connectivity to enable it to achieve greater value and service by exchanging data with the manufacturer, operator and/or other connected devices; each thing is uniquely identifiable through its embedded computing system but is able to interoperate within the existing Internet infrastructure. Internet Protocol version 6 (IPv6) is the most recent version of the Internet Protocol (IP), the communications protocol that provides an identification and location system for computers on networks and routes traffic across the Internet. See, e.g., Wikipedia. Although IPv6 and the IoT tend to be associated with physical non-living objects, there exists a need to associate or otherwise add a living element to the IoT and the various communication networks that implement the IPv6 protocol.

### SUMMARY

In accordance with some embodiments of the present disclosure, systems and methods relate to creating an environment having a holistic/intrinsic relationship that interchanges DNA genetic code with an Internet Protocol version 6 (IPv6) address. Such a relationship with a specific genetic code may be represented by a specific IP address, whereas a specific IP address equates to a specific DNA sequence. In accordance with some embodiments of the present disclosure, systems and methods generate IP address information, such as an IPv6 address itself, into a DNA strand and further utilize a formula, or algorithm, to transform a genetic address into a specific IP address. Accordingly, a mutual relationship between DNA code and IP addresses is created, as the DNA code, or sequence of nucleotides, identifies an individual and the DNA code is correlated to specific IPv6 addresses. The generation of an IP address into the DNA may then define a digital presence of a living thing. Essentially, a network element having location and traceability is created where individuals are both genetic and electronic endpoints existing in a symbiotic relationship.

Databases may contain both the genetic and electronic information of an individual. That is, the databases may contain DNA based on DNA sampling, IP information, or biometrics. The databases may be filled with information to identify individuals, but may also contain additional information about the individual. For example, the database may contain information associated with the individual, i.e. family, health, location, finances. The database may be the link to the individual and provide a transparent flow of information. These and other advantages will be apparent from the disclosure of the invention(s) contained herein.

In accordance with at least one aspect of the current disclosure, network endpoints utilizing a DNA and IP relationship may be provided. In essence, individuals having an identified unique IP address, as defined by their individual DNA, will be transparent to a “network”.

In accordance with at least some embodiments of the present disclosures, one or more algorithms or formulas are utilized to transform DNA sequence, or code, into an IPv6 address and then back again into the DNA sequence. In some embodiments there is very little variation or alteration in the algorithm/formula such that a DNA sequence can be determined based on an assigned IP address. That is, an individual's DNA sequence may be derived based on their assigned IP address. Similarly, an IP address may define an individual's DNA. Thus, DNA can be synthesized by virtue of the IP address of an organism.

In accordance with some embodiments of the present disclosure, an IP address may be used to determine a new or existing DNA sequence. For example, DNA according to an IP address may be synthesized from base pairs and associated with an existing DNA sequence. That is, by establishing a unique IP address associated with the genetic code, the flow of information and data accumulation may be seamless.

According to some embodiments of the present disclosure, a method is provided, the method comprising obtaining a DNA sequence, transforming the DNA sequence into an Internet Protocol (IP) address, and utilizing the IP address, accessing information associated with the IP address from a resource.

And yet in some embodiments, a system for converting a DNA sequence associated with a user into an Internet Protocol (IP) address is provided. The system may comprise a processor, memory, and a DNA/IP converter, wherein the DNA-IP converter obtains a DNA sequence, transforms the DNA sequence into an Internet Protocol (IP) address, and accesses information associated with the IP address from a resource.

And further yet, a method for encoding an Internet Protocol (IP) address into a DNA sequence to digitally define a living organism is provided, the method comprising obtaining an IP address, transforming the IP address into a DNA sequence, and synthesizing the DNA sequence, wherein the DNA sequence corresponds to the IP address.

The above-described embodiments, objectives, and configurations are neither complete nor exhaustive. As will be appreciated, other embodiments of the invention are possible using, alone or in combination, one or more of the features set forth above or described in detail below. Further, the summary of the invention is neither intended nor should it be construed as being representative of the full extent and scope of the present invention.

The present invention is set forth in various levels of detail in the summary of the invention, as well as in the attached drawings and the detailed description of the invention and no limitation as to the scope of the present invention is intended to either the inclusion or non-inclusion of elements, components, etc. in this summary of the invention. Additional aspects of the present invention will become more readily apparent from the detailed description, particularly when taken together with the drawings.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material”.

The term “computer-readable medium” as used herein refers to any tangible storage that participates in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, or any other medium from which a computer can read. Similarly, an in accordance with the embodiments of the present disclosure, DNA may be a form of computer-readable medium. When the computer-readable media is configured as a database, it is to be understood that the database may be any type of database, such as relational, hierarchical, object-oriented, and/or the like. Accordingly, the invention is considered to include a tangible storage medium and prior art-recognized equivalents and successor media, in which the software implementations of the present invention are stored.

The terms “determine”, “calculate”, and “compute,” and variations thereof, as used herein, are used interchangeably and include any type of methodology, process, mathematical operation or technique.

The term “module” as used herein refers to any known or later developed hardware, software, firmware, artificial intelligence, fuzzy logic, Network Functionality Virtualization (NFV) or combination of hardware and software that is capable of performing the functionality associated with that element. Also, while the invention is described in terms of exemplary embodiments, it should be appreciated that individual aspects of the invention can be separately claimed.

### BRIEF DESCRIPTION OF THE DRAWINGS

Those of skill in the art will recognize that the following description is merely illustrative of the principles of the disclosure, which may be applied in various ways to provide many different alternative embodiments. It should be understood that the drawings are not necessarily to scale and in certain instances, details that are not necessary for an understanding of the disclosure or that render other details difficult to perceive may have been omitted.

FIG. 1 depicts details of a DNA sequence and an IPv6 address in accordance with embodiments of the present disclosure (SEQ ID NO:2);

FIG. 2 depicts an example communication network wherein each item of the network may identify, locate, and communicate, in some manner, using an IP address of the organism in accordance with embodiments of the present disclosure;

FIG. 3A depicts an example transformation of a DNA sequence belonging to an individual into an IPv6 address in accordance with embodiments of the present disclosure;

FIG. 3B depicts an example transformation of an IPv6 address into a DNA sequence in accordance with embodiments of the present disclosure;

FIG. 4 depicts an example transformation of an organism's DNA sequence into an IP address utilizing an algorithm/formula/transformation, the IP address is then transformed back into a DNA sequence to be stored within an Organism B in accordance with embodiments of the present disclosure;

FIG. 5 depicts an example transformation of data and an IP address being transformed into one or more sequences of DNA to be stored within an organism in accordance with embodiments of the present disclosure;

FIG. 6 depicts details of a DNA/IP converter in accordance with embodiments of the present disclosure;

FIG. 7 depicts a first flow diagram depicting an example transformation of DNA into an IPv6 address in accordance with embodiments of the present disclosure; and

FIG. 8 depicts a second flow diagram depicting an example transformation of an IPv6 address into DNA in accordance with embodiments of the present disclosure.

### DETAILED DESCRIPTION

The ensuing description provides embodiments only, and is not intended to limit the scope, applicability, or configuration of the claims. Rather, the ensuing description will provide those skilled in the art with an enabling description for implementing the embodiments. It being understood that various changes may be made in the function and arrangement of elements without departing from the spirit and scope of the appended claims.

The present invention has significant benefits across a broad spectrum of endeavors. It is the applicant's intent that this specification and the claims appended hereto be accorded a breadth in keeping with the scope and spirit of the invention being disclosed despite what might appear to be limiting language imposed by the requirements of referring to the specific examples disclosed. To acquaint persons skilled in the pertinent arts most closely related to the present invention, a preferred embodiment of the method that illustrates the best mode now contemplated for putting the invention into practice is described herein by, and with reference to, the annexed drawings that form a part of the specification. The exemplary method is described in detail without attempting to describe all of the various forms and modifications in which the invention might be embodied. As such, the embodiments described herein are illustrative, and as will become apparent to those skilled in the arts, can be modified in numerous ways within the scope and spirit of the invention.

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning.

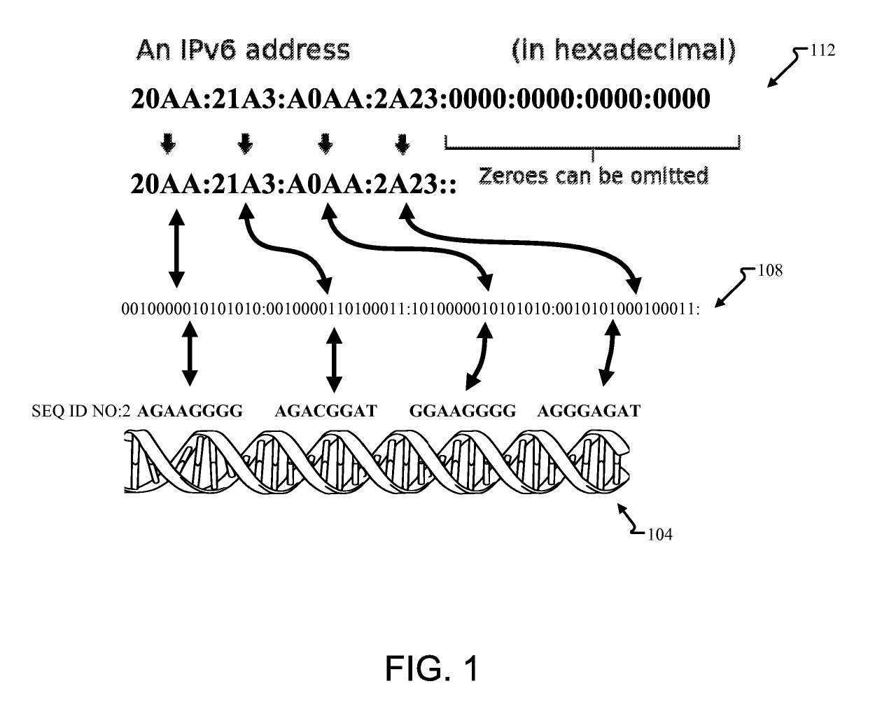

FIG. 1 depicts an example of an IPv6 address and associated DNA sequence in accordance with embodiments of the present disclosure. An Internet Protocol Version 6 address (IPv6 address) is an alpha-numeric, or textual, label that is used to identify a network interface of a computer or other network node participating in an IPv6 network. IPv6 was originally developed by the Internet Engineering Task Force (IETF) to deal with the long-anticipated problem of IPv4 address exhaustion. IPv6 addresses are represented as eight groups of four hexadecimal digits separated by colons, for example 2001:0DB8:85A3:0042:1000:8A2E:0370:7334, but methods to abbreviate this full notation exist. For example, as illustrated in FIG. 1, the fully qualified IPv6 address having eight groups of four hexadecimal digits separated by colons can also be represented by compressing and/or eliminating long strings of zeros. Accordingly, the IPv6 address of 20AA:21A3:AOAA:2A23:0000:0000:0000:0000 may be compressed to 20AA:21A3:AOAA:2A23::, where the colon represents a string of zeros. In accordance with rules set forth by the IETF, some strings of zeros may be eliminated while others must remain. Internet Protocol version 6 (IPv6) represents a newer communications protocol that provides identification and location systems for “computers” on networks and routes traffic based on a computer's identification and location. Internet Protocol (IP) essentially is a marker used for identification and location in the digital language.

Deoxyribonucleic acid (DNA) is also illustrated in FIG. 1; DNA is a molecule that encodes the genetic instructions used in the development and functioning of all known living organisms and many viruses. One of DNA's purposes is to transmit information, which is encoded in chemical language. In accordance with at least one embodiment of the present disclosure, these two “elements” (DNA and IPv6) are mutually connected by establishing a link or translation between the digital language and the chemical language. The result is that DNA and IPv6 are tied together having a mutual existence for the purpose of storage, transport, and identification.

A single strand of DNA contains 4 nucleotides: Guanine, Adenine, Thymine and Cytosine. The combination of these nucleotides makes up the genetic code of any living organism. (GATC in short form). A linkage between location and identification may be established utilizing an algorithm and/or decipher program implemented on one or more hardware elements. By taking the four nucleotides, A, T, G and C and digitizing, or creating a combination of 1's and 0's for example as a representative of the nucleotide, such a linkage may be established.

Identification techniques may rely on DNA profiles and/or DNA sequences. Similar to fingerprints, every individual possesses their own unique DNA sequence. Therefore, a genetic profile may include a unique DNA sequence that is exclusive to that person. To generate a genetic profile, a sample of DNA is first obtained. Real-time polymerase chain reaction (PCR) may then be used to detect and quantify an amount of available DNA from the sample. Specific portions of the DNA are then copied and created using a process called amplification; accordingly, PCR greatly amplifies the amounts of a specific region, or portion, of DNA. In another technique, gel electrophoresis may be utilized; gel electrophoresis separates the different DNA portions according to size. The DNA sample may then be searched for specific segments of DNA that repeat themselves. Although 99% of human DNA is shared, segments, called Short Tandem Repeats (STR), may be unique to individuals. That is, although humans share over 99% of their DNA, every individual has a particular set of STR markers; these STR markers are inherited from different genes from each parent. Accordingly, the chance of two unrelated people having the same pattern is very low.

Alternatively, or in addition, and in accordance with embodiments of the present disclosure, a sample of DNA may be obtained and sequenced; non-liming methods of obtaining the DNA sample include orally by dry, wet, and/or non-invasive oral procedures such as collecting saliva, or the DNA may be obtained from other sources, such as hair with follicle, blood, urine, sweat or other body fluids. DNA sequencing is the process of determining the order of nucleotides within a DNA molecule. DNA sequencing includes any method or technology that is used to determine the order of the four bases—adenine, guanine, cytosine, and thymine—in a strand of DNA.

In accordance with embodiments of this disclosure, the same or similar techniques for obtaining a sample of DNA and generating a DNA profile and/or DNA sequence may be utilized; however, such process becomes unique in that the profile and/or unique strand of DNA base pairs is utilized to generate a unique IPv6 address. That is, an IPv6 address may then be generated based on the sequence of nucleotides within DNA as described herein. Such conversion may rely on a text/binary/hex conversion algorithm.

In accordance with some embodiments of the present disclosure, suppose the nucleotides A, T, G, and C are mapped to a binary representation as shown in Table 1.

TABLE 1

| Nucleotide | Binary Representation |
|---|---|
| Adenine (A) | 01000001 |
| Cytosine (C) | 01000011 |
| Guanine (G) | 01000111 |
| Thymine (T) | 01010100 |

A typical DNA sequence obtained by conventional sampling methods may be as follows: ATCGATTGAGCTCTAGCG (SEQ ID NO:1). Accordingly, the DNA sequence may be converted to binary format by translating the DNA sequence into a binary format based on the mapping shown in Table 1. The resulting binary sequence of 1's and 0's based on the DNA sequence is shown in Table 2.

TABLE 2

| A | T | C | G | A | T |
|---|---|---|---|---|---|
| 01000001 | 01010100 | 01000011 | 01000111 | 01000001 | 01010100 |
| T | G | A | G | C | T |
| 01010100 | 01000111 | 01000001 | 01000111 | 01000011 | 01010100 |
| C | T | A | G | C | G |
| 01000011 | 01010100 | 01000001 | 01000111 | 01000011 | 01000111 |

The resulting binary sequence of: 010000010101010001000011010001110100000101010100010101000100011101000001010001 110100001101010100010000110101010001000001010001110100001101000111 may be converted into hexadecimal format for use by an IPv6 address as defined by the Internet Engineering Task Force (IETF). The hexadecimal conversion of the binary equivalent of the first eight DNA nucleotides then becomes: 20AA21A3A0AA2A23, which may be parsed into octets resulting in an equivalent IPv6 address of 20AA:21A3:AOAA:2A23::.

As another example, and in accordance with some embodiments of the present disclosure, suppose the nucleotides A, T, G, and C are mapped to a binary representation as shown in Table 3.

TABLE 3

| Nucleotide | Binary Representation |
|---|---|
| Adenine (A) | 00 |
| Cytosine (C) | 01 |
| Guanine (G) | 10 |
| Thymine (T) | 11 |

A typical thirty-two character DNA sequence 104 obtained by conventional sampling methods may be as follows: AGAAGGGGAGACGGATGGAAGGGGAGGGAGAT (SEQ ID NO:2). Accordingly, the DNA sequence 104 may be converted to binary format 108 by translating the DNA sequence 104 into a binary format 108 based on the mapping shown in Table 3. The resulting binary sequence 108 of 1's and 0's based on the DNA sequence 104 is shown in Table 4.

TABLE 4

| A | G | A | A | G | G | G | G | A | G | A | C | G | G | A | T |
|---|---|---|---|---|---|---|---|---|---|---|---|---|---|---|---|
| 00 | 10 | 00 | 00 | 10 | 10 | 10 | 10 | 00 | 10 | 00 | 01 | 10 | 10 | 00 | 11 |
| G | G | A | A | G | G | G | G | A | G | G | G | A | G | A | T |
| 10 | 10 | 00 | 00 | 10 | 10 | 10 | 10 | 00 | 10 | 10 | 10 | 00 | 10 | 00 | 11 |

The resulting binary sequence 108 of: 0010000010101010:0010000110100011:1010000010101010:0010101000100011 may be converted into a hexadecimal format for use by an IPv6 address as defined by the Internet Engineering Task Force (IETF). For example, the nucleotide sequence of AG in binary is 0010; 0010 in hexadecimal is 2. Similarly, the nucleotide sequence of GG is 1010; 1010 in hexadecimal is A. A hexadecimal equivalent of the binary sequence may then become: 20AA21A3A0AA2A23 which may be parsed into octets resulting in an equivalent IPv6 address 112 of 20AA:21A3:AOAA:2A23::.

As another example, a typical sixty-four character DNA sequence obtained by conventional sampling methods may be as follows: AGAAGGGGAGACGGATGGAAGGGG AGGGAGATGCACAATGGGATCATGGAAATCCCATGGACTA (SEQ ID NO:3).

Accordingly, the DNA sequence may be converted to binary format by translating the DNA sequence into a binary format based on the mapping shown in Table 3. The resulting binary sequence of 1's and 0's based on the DNA sequence is shown in Table 5.

TABLE 5

| A | G | A | A | G | G | G | G | A | G | A | C | G | G | A | T |
|---|---|---|---|---|---|---|---|---|---|---|---|---|---|---|---|
| 00 | 10 | 00 | 00 | 10 | 10 | 10 | 10 | 00 | 10 | 00 | 01 | 10 | 10 | 00 | 11 |
| G | G | A | A | G | G | G | G | A | G | G | G | A | G | A | T |
| 10 | 10 | 00 | 00 | 10 | 10 | 10 | 10 | 00 | 10 | 10 | 10 | 00 | 10 | 00 | 11 |
| G | C | A | C | A | A | T | G | G | G | A | T | C | A | T | G |
| 10 | 01 | 00 | 01 | 00 | 00 | 11 | 10 | 10 | 10 | 00 | 11 | 01 | 00 | 11 | 10 |
| G | A | A | A | T | C | C | C | A | T | G | G | A | C | T | A |
| 10 | 00 | 00 | 00 | 11 | 01 | 01 | 01 | 00 | 11 | 10 | 10 | 00 | 01 | 11 | 00 |

The resulting binary sequence of: 001000001010101000100001101000111010000010101000101010001000111001000100001110 101000000100111010000000110101010011101000011100 may be converted into a hexadecimal format for use by an IPv6 address as defined by the Internet Engineering Task Force (IETF). Such conversion may obtain a hexadecimal character for each two nucleotide sequence. For example, the nucleotide sequence of AG in binary is 0010; 0010 in hexadecimal is 2. Similarly, the nucleotide sequence of GG is 1010; 1010 in hexadecimal is A. A hexadecimal equivalent of the binary sequence may then become: 20AA21A3A0AA2A23910EA34E80D53A1C which may be parsed into octets resulting in an equivalent IPv6 address of 20AA:21A3:AOAA:2A23:910E:A34E:80D5:3A1C.

In accordance with at least some embodiments of the present disclosure, a DNA sequence may be obtained based on an IPv6 address. For example, an IPv6 address may be converted directly into hexadecimal by removing the colons. Accordingly, a binary equivalent of 20AA1A3A0AA2A23 is illustrated in Table 6.

TABLE 6

| A | T | C | G | A | T |
|---|---|---|---|---|---|
| 01000001 | 01010100 | 01000011 | 01000111 | 01000001 | 01010100 |
| T | G | A | G | C | T |
| 01010100 | 01000111 | 01000001 | 01000111 | 01000011 | 01010100 |
| C | T | A | G | C | G |
| 01000011 | 01010100 | 01000001 | 01000111 | 01000011 | 01000111 |

Thus, the IPv6 address may be expressed as a DNA sequence of ATCGATTGAGCTCTAGCG. Clearly the combinations of bits, bytes, IP addresses, and DNA are endless; that is, there are efficient ways to digitally represent the combination of nucleotides utilizing a digital representation.

In addition, the IEEE, or governing bodies may allocate a block of designated IPv6 addresses for human or any living organism classification. The methodology herein encompasses the means of conversion of nucleotides to IPv6 address which may be dependent upon agreed or authorized convention—thus, the process may vary depending on acceptance criteria lawfully in effect. The particular conversion process, as outlined above, may convert a textual DNA sequence of A, C, T, and Gs into a binary representation, which is then converted into a hexadecimal representation. Alternatively, or in addition, the textual DNA sequence of A, C, T, and Gs is directly converted into a hexadecimal representation.

In accordance with at least one embodiment of the present disclosure, the role of Internet Protocol, or IPv6, may be used for identification and transport of information in a network/Internet world. Utilizing a DNA/IP relationship creates a number of different roles for IP in a DNA world. For example, in the context of transport, DNA may be utilized for digital storage; inevitably there is a need for a source and destination to be embedded within the digital content in front of the media encoded in the DNA. An encoded IPv6 address in front of the stored media, which is also encoded in DNA, may be used for a number of things. For example, the encoded IPv6 address may be used as an identifier of the source and destination and/or the rules by which the media is sent through a network to a specific identifier of the media or content itself, for example, the genetic code of a person. Moreover, such transport based on the IPv6 address may coincide or otherwise be performed in accordance with the Internet Protocol, Version 6 (IPv6) Specification RFC 2460 published in December 1998, which is incorporated herein by reference for all that it teaches and for all purposes. In the context of storage, an IPv6 address may be used in a number of ways. Similar to transport, for example, the IPv6 address may provide the content identification, be the identifier of the content, and/or be utilized as the source and destination of the content that is stored.

In the context of identification, the ability to have a unique IP address associated with an individual and directly correlating the IP address to the individual's genetic code provides a method of identification. For example, a DNA-IP linkage may be used for military purposes, healthcare, finances, finding lost children, etc. Accordingly, an individual may be effectively connected to the world; that is, the individual is likened to a network element with not only a genetic identifier, but a digital one.

In accordance with some embodiments of the present disclosure, potential uses will coexist with centralized, secure database(s). The mode of interconnection to these databases may operate in a number of ways, be it webpage based portals, VPN, SDN, or other secure software based models. Or, such uses may essentially evolve with the ever-changing dynamics of the network themselves. The critical aspect with respect to data will be the security of the connection and the robust nature of secure applications utilizing or sampling the data. Potential uses for a DNA-IP linkage may include, but are not limited to, the following:

Law Enforcement—Tracking of human beings over the global network infrastructure. Convicted criminals or prisoners may be tracked for life everywhere and anywhere. Accordingly, the DNA-IP linkage may facilitate automatic lockdowns of prisoners in half-way homes or on parole. Secondary offenders may be tracked and captured more easily via triangulation and GPS.

Defense—Control of soldier populations, troop tracking, digital records retention, friendly fire confirmations, etc. For example, a soldier may not only be identified, but may also be located based on the DNA-IP linkage.

Medical—As one example, a patient may enter a hospital and one or more charts that include medical information associated with the patient are automatically updated via their presence. In some instances, the IP address associated with the patient's DNA may be utilized to access additional information, or records, containing information concerning the patient.

Government—SIN numbers, tax laws tracking, census counts, etc. via human recognition.

Financial—In some instances, an IPv6 address linked to personal banking information may be utilized. Accordingly, debit cards and credit cards may no longer required. In addition, the valuation of a person may occur upon entry into a store, banking institution, etc.

Workplace—In some instances, an employee may be tracked, for example when the employee enters a work area. As such, the DNA-IP linkage may be utilized for timecard tracking, payroll, etc.

Social—Essentially, an individual may be linked to a webpage. That is, the DNA-IP linkage may provide for real-time tracking and updating of activities, moods, thoughts etc. In accordance with at least some embodiments, if an individual were to take a photographic image, the image may be accessible via the IP address associated with the individual's DNA.

In accordance with some embodiments of the present disclosure, a person can function as an “end point” of a computer network, by virtue of their genetic code—IPv6 address. From a practical standpoint, having the ability to associate an individual's or organism's genetic code with a specified IP (IPv6) address enables the possibility for individuals to essentially be “web pages.” That is, a unique DNA string makes it the “holy grail” of identification. With the concept of a web page for an individual, the applications become endless. From a page popping up at a country's border when a person enters customs, to law enforcement & military uses, to health care fields, financial, and the list goes on.

Due to the nature of the IPv6/DNA relationship as described herein, a person's IPv6 address and associated web page may be intertwined. As the person moves within a network structure, the webpage may be updated with location information. If a person goes to a restaurant, spends money, and then buys a ticket for a show, the webpage can essentially reflect these events as they are intertwined and the information for each of these events resides within the IPv6 node or web page.

In accordance with some embodiments, proponents of utilizing a DNA-IPv6 link include security proponents and moralistic/big brother proponents. The idea of having a “singular” type of control like an IP address linking to a “web page” or as “defined by the standards or governing bodies” is that security will be far easier to control. For example, it is easier and more efficient to protect a single pin hole, as opposed to multiple holes. Accordingly, an individual's security and private data may be controlled by a single path in-route to a field of customized “web type” pages. Governing bodies may have the ultimate control as to how this data is utilized; but, more importantly, the ultimate control to protecting the data.

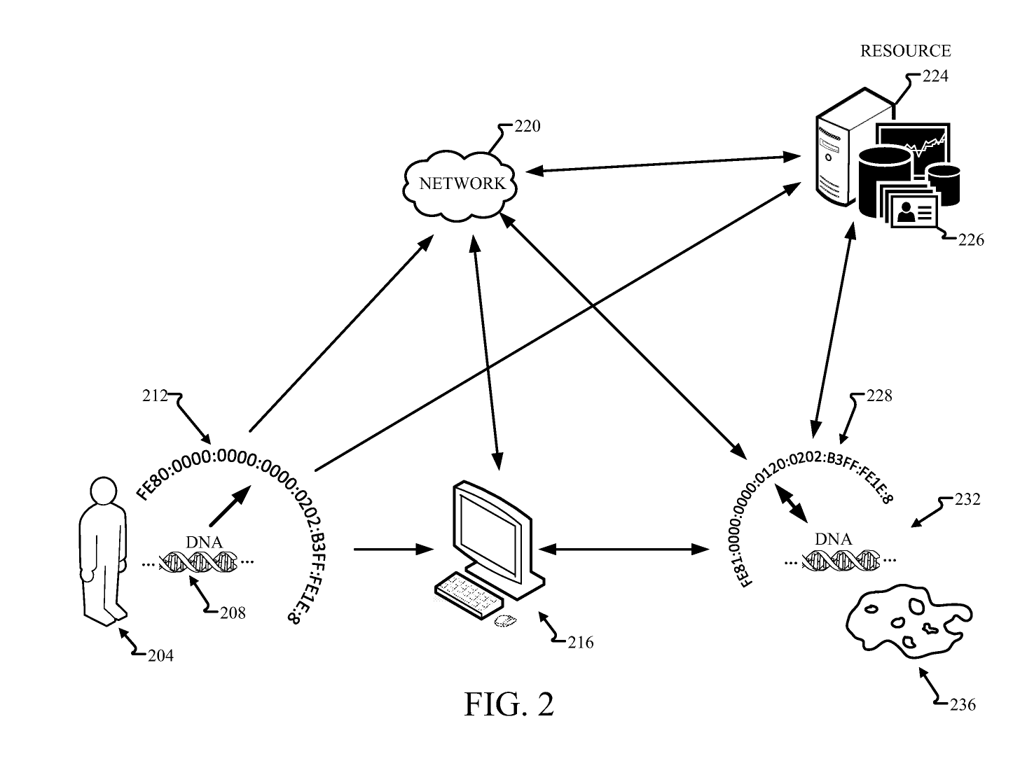

FIG. 2 depicts an exemplary communication network according to at least one embodiment of the present disclosure. As illustrated in FIG. 2, a communication network may include an individual such as an individual 204 having a DNA sequence 208 and associated IP address 212, a communication network 220, a computing device 216, a resource 224, and an organism 236 other than the individual 204, where the organism 236 has a unique DNA sequence 232 and an associated IP address 228. In accordance with some aspects of the present disclosure, the individual 204 may communicate with a computing device 216, resource 234, or other organism 236 either directly or via the communication network 220. That is, an individual may provide a DNA sequence 208 which is transformed in a unique IP address 212. Upon detecting, or otherwise receiving, the unique IP address 212, the computing device 216, resource 234, and/or other organism 236 may perform some function. In at least one embodiment, the DNA sequence may be created by obtaining a sample of DNA and sequencing the sample to generate a portion of the individual's DNA sequence, such portion may then be converted into an IPv6 address. For example, as the user 204 walks into a hospital, a sample or detected instance of the individual's DNA sequence 208 may be converted into an IP address 212; this IP address 212 may then allow the computing device 216 and/or resource 224 to identify, locate, and retrieve medical records or other such information 226 associated with the individual 204. Such information 226 may be stored within a database of resource 224; alternatively, or in addition, resource 224 may be a database. In some instances, and utilizing the IP address 228 of an organism 236, the IP address 212 associated with the individual 204 may be provided, along with other optional data, to the organism 236 such that the organism 236 may store information, such as information associated with the user 236, in a DNA sequence.

The communication network 220 may be packet-switched and/or circuit-switched. An illustrative communication network 220 includes, without limitation, a Wide Area Network (WAN), such as the Internet, a Local Area Network (LAN), a Personal Area Network (PAN), a Public Switched Telephone Network (PSTN), a Plain Old Telephone Service (POTS) network, a cellular communications network, an IP Multimedia Subsystem (IMS) network, a Voice over IP (VoIP) network, a SIP network, or combinations thereof. The Internet is an example of the communication network 220 that constitutes an Internet Protocol (IP) network including many computers, computing networks, and other communication devices located all over the world, which are connected through many communication systems and other means. In one configuration, the communication network 220 is a public network supporting the TCP/IP suite of protocols. Communications supported by the communication network 220 include real-time, near-real-time, and non-real-time communications. For instance, the communication network 220 may support voice, video, text, web-conferencing, or any combination of media and may facilitate one or more signaling protocols to set up, maintain, and/or tear down a communication session, communication thread, communication flow, and the like. Moreover, the communication network 220 may comprise a number of different communication media, such as coaxial cable, copper cable/wire, fiber-optic cable, antennas for transmitting/receiving wireless messages, and combinations thereof. It can be appreciated that the communication network 220 need not be limited to any one network type, and instead may be comprised of a number of different networks and/or network types. In particular, and as described herein, the communication network 220 may be capable of routing IPv6.

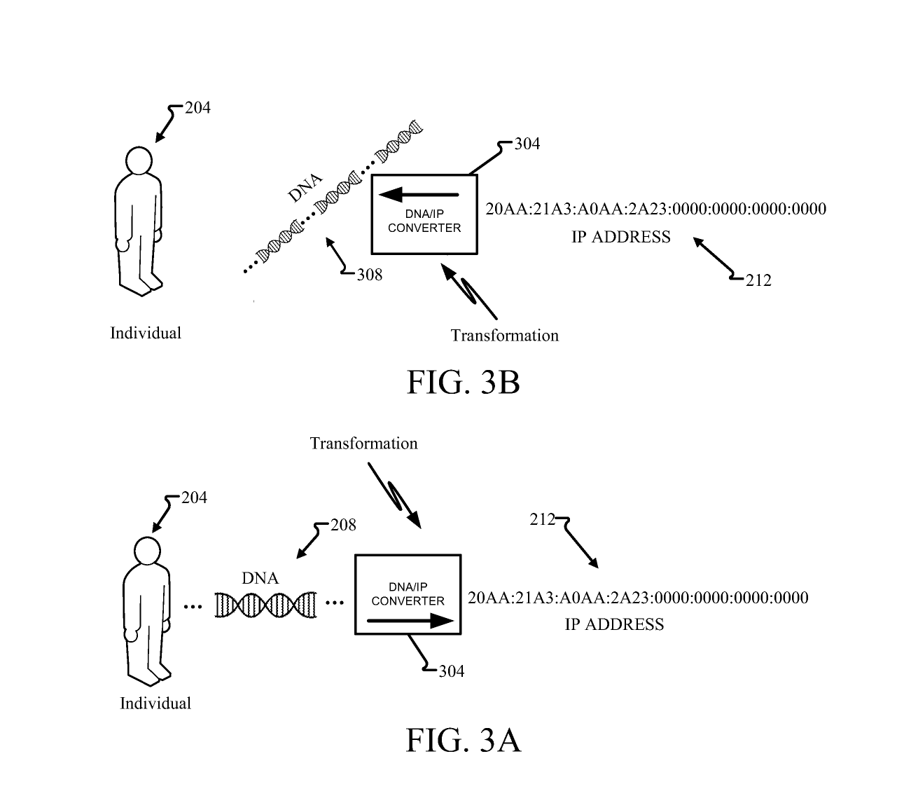

FIGS. 3A-3B depict one or more transformations of a DNA sequence in accordance with at least some embodiments of the present disclosure. As illustrated in at least FIG. 3A, DNA sequence 208 associated with an individual 204 is transformed into an IP address 212. Such a transformation may utilize the DNA/IP converter 304 to make such a transformation. For example, and as will be described later, the DNA/IP converter 304 may include one or more algorithms that directly convert a DNA sequence 208 into an IP address 212. In some instances, such an algorithm may include one-way and/or two-way hashes such that a unique IP address 212 is generated based on a unique DNA sample or DNA sequence 208. As illustrated in at least FIG. 3B, an IP address 212 may be converted directly into a DNA sequence 308 using the DNA/IP converter 304. That is, the DNA/IP converter may include the necessary functionality to not only convert DNA into an IP address, but also convert an IP address into DNA. Similar to the DNA to IP conversion, the DNA/IP converter may rely upon a specific process or algorithm to convert the IP address into DNA. In some instances, such an algorithm may include one-way and/or two-way hashes such that a unique IP address 212 generates a unique DNA sequence.

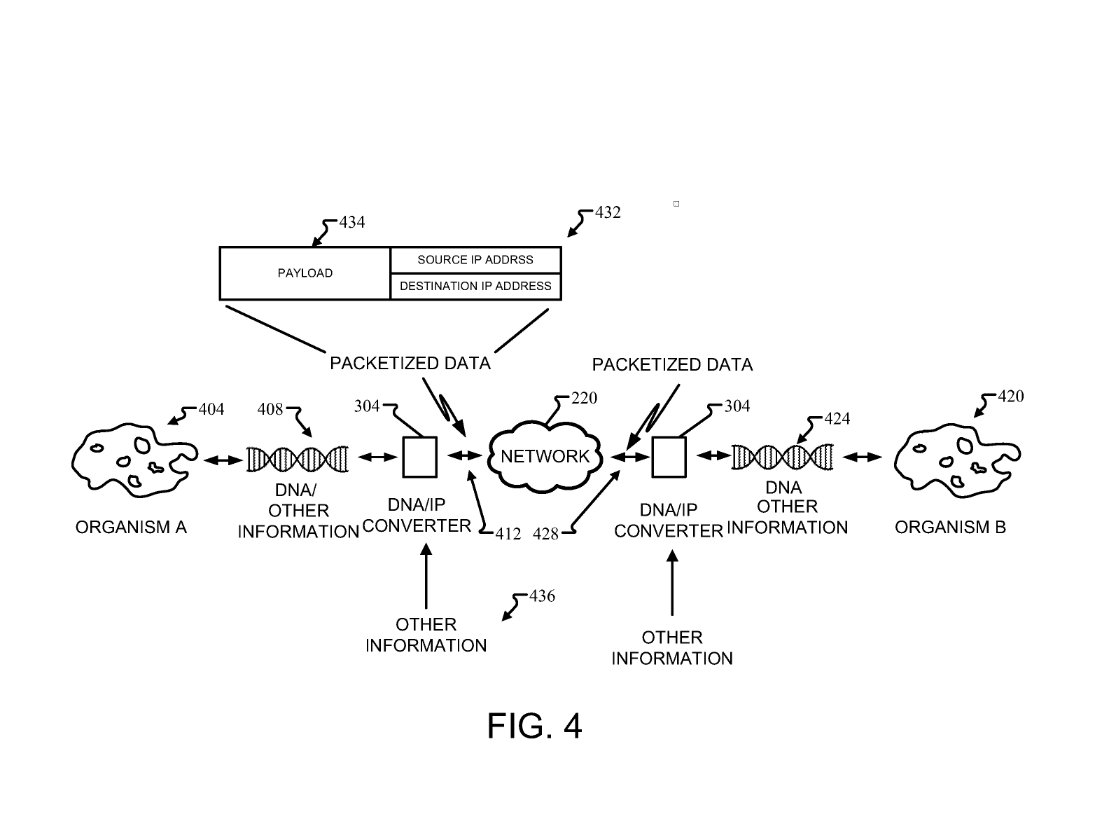

In accordance with at least some embodiments of the present disclosure, FIG. 4 illustrates transportation of information utilizing an IP-DNA relationship. For example, an Organism A's 404 DNA sequence 408 may be converted into an IP address 412 utilizing the previously described DNA/IP converter 304, which may utilize an algorithm and/or formula. Additionally, an Organism B's 420 DNA sequence 424 may be converted into an IP address 428 utilizing the previously described DNA/IP converter 304, which may utilize an algorithm and/or formula. Other information, such as other information 436 or information that may be included in a sequence of Organism's A 404 DNA sequence 408 may be converted to a form of electronic data and included in a payload 434 such that in combination with the IP addresses 412, 428 of Organism A 404 and Organism B 420 respectively, the other information 436, in electronic data form, such as a packetized payload 434, may be transported or otherwise communicated across the communication network 220 as packetized data 432. Once communicated to an intended location, the information, including the IP address 412, 428 of Organism A 404 and Organism B 420 respectively, may be converted into a DNA sequence utilizing an algorithm and/or formula within the DNA/IP converter 304. Utilizing the IP address converted to DNA, the other information 436 may be stored at Organism B 420. In at least one embodiment consistent with the present disclosure, Organism A 404 may communicate with Organism B 420 and vise-versa utilizing their respective IP addresses based on their respective DNA sequences.

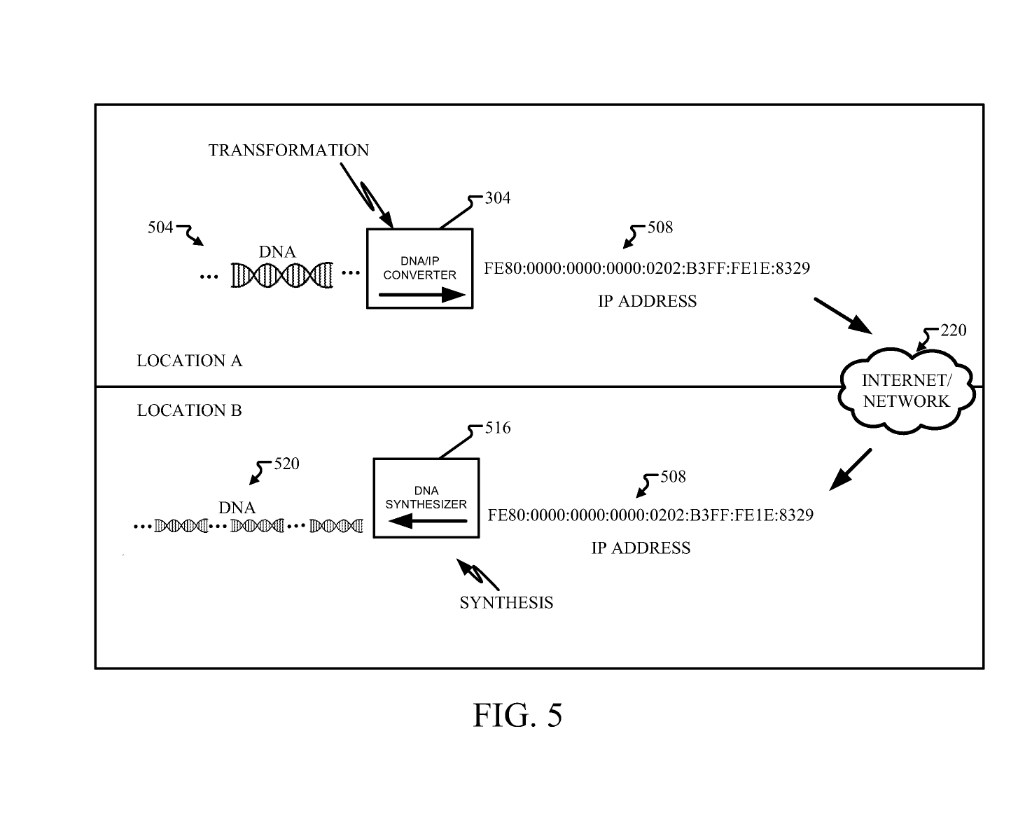

FIG. 5 depicts an exemplary embodiment for making, or otherwise synthesizing, DNA from an IP address. In some embodiments, DNA 504 may be first digitized for transport, transported across a communication network 220 from location A to another location B, and then synthesized at location B. For example, and in accordance with at least one embodiment of the present disclosure, a DNA sequence 504 may be transformed into an IP address 508 as illustrated. The IP address 508, and any other data associated with the IP address 508, such as other information 436, may then be transported to location B. At location B, the IP address 508 may then be synthesized into DNA 520. As one example, the IP address 508 may be first converted to a digital sequence of DNA 520 representing nucleotide pairs and then synthesized into actual DNA nucleotides; that is, the DNA sequence 520 may be synthesized into a nucleic acid and/or short fragments of a nucleic acid. Such synthesis may be performed utilizing existing oligonucleotide synthesis and artificial gene synthesis techniques, but other techniques are contemplated.

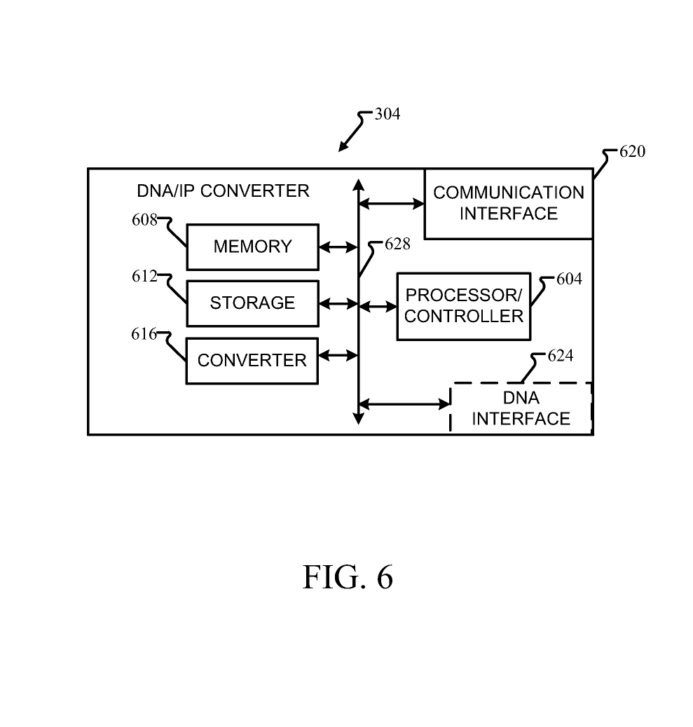

FIG. 6 depicts additional details of the DNA/IP Converter 304 in accordance with some embodiments of the present disclosure. In some embodiments, the DNA/IP Converter 304 may reside within, or be part of a computing device 216 and/or a resource 224. The DNA/IP Converter 304 may generally include a processor/controller 604, memory 608, storage 612, a converter 616, a communication interface 620, and an optional DNA interface 624. The processor/controller 604 is provided to execute instructions contained within memory 608. Accordingly, the processor/controller 604 may be implemented as any suitable type of microprocessor or similar type of processing chip, such as any general-purpose programmable processor, digital signal processor (DSP) or controller for executing application programming contained within memory 608. Alternatively, or in addition, the processor/controller 604 and memory 608 may be replaced or augmented with an application specific integrated circuit (ASIC), a programmable logic device (PLD), or a field programmable gate array (FPGA).

The memory 608 generally comprises software routines facilitating, in operation, pre-determined functionality of the DNA/IP Converter 304. The memory 608 may be implemented using various types of electronic memory generally including at least one array of non-volatile memory cells (e.g., Erasable Programmable Read Only Memory (EPROM) cells or FLASH memory cells, etc.) The memory 608 may also include at least one array of dynamic random access memory (DRAM) cells. The content of the DRAM cells may be pre-programmed and write-protected thereafter, whereas other portions of the memory may selectively be modified or erased. The memory 608 may be used for either permanent data storage or temporary data storage. Alternatively, or in addition, data storage 612 may be provided. The data storage 612 may generally include storage for programs and data. For instance, and with respect to the DNA/IP Converter 304, data storage 612 may provide storage for the DNA/IP Converter 304 to store DNA sequences, IP addresses, one or more conversion tables for use by the converter 616 in transforming DNA into IP addresses or IP addresses into DNA. The converter 616 may convert or otherwise transform one or more sequences of DNA, as previously discussed, into one or more IP addresses by implementing one or more conversion formulas, functions, and/or algorithms. The DNA/IP Converter 304 may further include a DNA interface 624; the DNA interface 624 may provide functionality to obtain a sample of DNA in accordance with previously described DNA sampling techniques. The DNA interface 624 may further provide the functionality to sequence at least a portion of the sample of DNA into a sequence of nucleotides. Alternatively, or in addition, the DNA interface 624 may provide the necessary functionality to synthesize one or more DNA sequences into DNA nucleotides in accordance with previously described DNA synthesis techniques. The communication interface 620 may provide then necessary functionality to communicate with the network 220 and/or receive one or more DNA sequences. Communications between various components of the DNA/IP Converter 304 may be carried by one or more buses 628.

Alternatively, or in addition, the computing device 216 and the resource 224 may include the DNA/IP Converter 304. Alternatively, or in addition, the computing device 216 and the resource 224 may include the processor/controller 604, memory 608, storage 612, and communication interface 620. Accordingly, the addition of the converter 616 and/or the optional DNA interface 624 to the computing device 216 and/or the resource 224, provides the computing device 216 and/or the resource 224 with the DNA/IP Converter 304 functionality described herein.

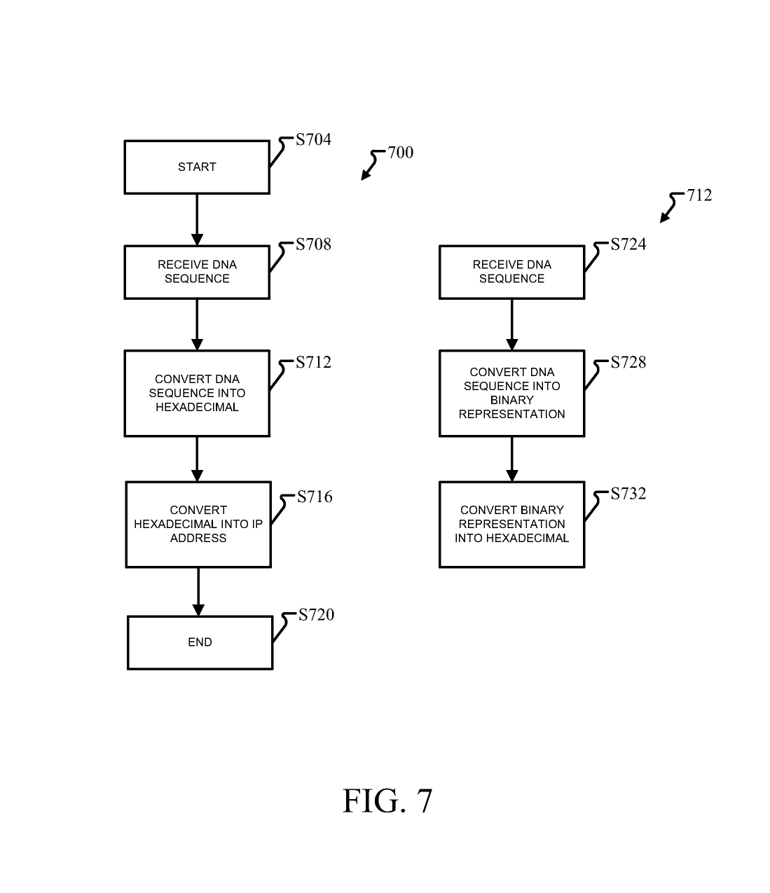

Referring now to FIG. 7, a method 700 of converting a DNA sequence into an IP address will be discussed in accordance with embodiments of the present disclosure. Method 700 is in embodiments, performed partially or wholly by a device, such as the DNA/IP converter 304. More specifically, one or more hardware and software components may be involved in performing method 700. In one embodiment, one or more of the previously described modules and/or devices perform one or more of the steps of method 700. The method 700 may be executed as a set of computer-executable instructions executed by a computer system or DNA/IP converter 304 and encoded or stored on a computer-readable medium. Hereinafter, the method 700 shall be explained with reference to systems, components, modules, software, etc. described with FIGS. 1-6.

Method 700 may continuously flow in a loop, flow according to a timed event, or flow according to a change in an operating or status parameter. Method 700 is generally initiated at step S704, where the DNA/IP converter 304 may initiate a DNA conversion process. Accordingly, at step S708, a DNA sequence may be received. Once the DNA sequence is received, the DNA/IP converter 304 may convert, at step S712, the received DNA sequence into hexadecimal form as previously discussed herein. Such hexadecimal form may then be converted, at step S716, into an IP address, such as an IPv6 address, wherein the IPv6 address is formatted for use within an IPv6 network. The method 700 may then end at step S720.

Additional details of the DNA to IP conversion process are described with respect to step 712. That is, as illustrated, once the DNA sequence is received at step S724, the DNA/IP converter 304 may convert the DNA sequence first into a Binary representation at step S728 as previously discussed. The Binary representation may then be converted to a hexadecimal representation at step S732.

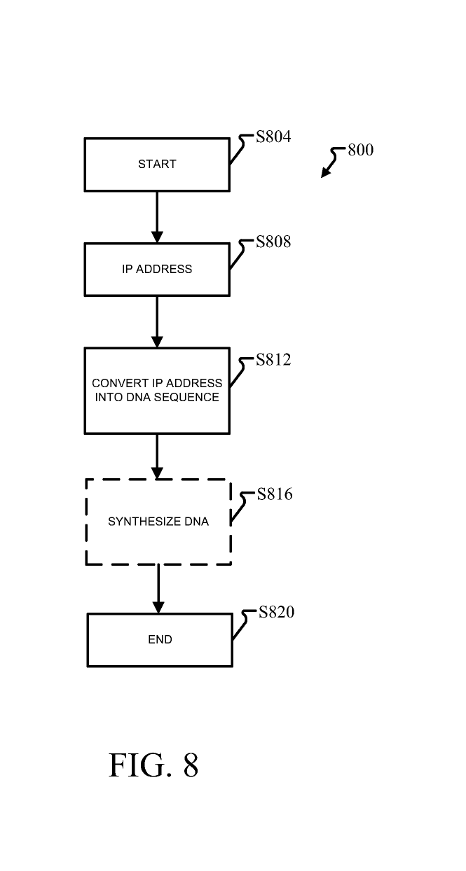

Referring now to FIG. 8, a method 800 of converting a DNA sequence into an IP address will be discussed in accordance with embodiments of the present disclosure. Method 800 is in embodiments, performed partially or wholly by a device, such as the DNA/IP converter 304. More specifically, one or more hardware and software components may be involved in performing method 800. In one embodiment, one or more of the previously described modules and/or devices perform one or more of the steps of method 800. The method 800 may be executed as a set of computer-executable instructions executed by a computer system or DNA/IP converter 304 and encoded or stored on a computer-readable medium. Hereinafter, the method 800 shall be explained with reference to systems, components, modules, software, etc. described with FIGS. 1-7.

Method 800 may continuously flow in a loop, flow according to a timed event, or flow according to a change in an operating or status parameter. Method 800 is generally initiated at step S804, where the DNA/IP converter 304 may initiate an IP conversion process. Accordingly, at step S808, an IP address may be received. Once the IP address is received, the DNA/IP converter 304 may convert, at step S812, the received IP address into a DNA sequence as previously discussed herein. At step S816, an optional step, the DNA sequence may be synthesized into DNA nucleotides as previously discussed. The method 800 may then end at step S820.

In the foregoing description, for the purposes of illustration, methods were described in a particular order. It should be appreciated that in alternate embodiments, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine-readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

Specific details were given in the description to provide a thorough understanding of the embodiments. However, it will be understood by one of ordinary skill in the art that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Also, it is noted that the embodiments were described as a process, which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a machine-readable medium, such as a storage medium. A processor(s) may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

While illustrative embodiments of the invention have been described in detail herein, it is to be understood that the inventive concepts may be otherwise variously embodied and employed, and that the appended claims are intended to be construed to include such variations, except as limited by the prior art.

What is claimed is:

1. A method for accessing information about an individual, the method comprising:
obtaining a DNA sequence;
transforming the DNA sequence into an Internet Protocol (IP) address; and
utilizing the IP address, accessing information associated with the IP address from a resource.

2. The method of claim 1, wherein the IP address is an Internet Protocol Version 6 (IPv6) address.

3. The method of claim 1, wherein the information associated with the IP address includes at least one of health information, location information, and financial information.

4. The method of claim 1, wherein the DNA sequence is unique for the user.

5. The method of claim 1, further comprising:
obtaining a destination IP address for the resource; and
packaging the destination IP address and the IP address transformed from the DNA sequence into a communication packet.

6. The method of claim 5, further comprising transmitting the communication packet utilizing a communication network, wherein the communication packet is transmitted to a location associated with the destination IP address.

7. The method of claim 1, further comprising:
receiving the IP address;
converting the IP address into a DNA sequence; and
synthesizing the DNA sequence into a nucleic acid molecule.

8. The method of claim 1, further comprising:
obtaining a DNA sample;
sequencing at least a portion of the DNA sample; and
transforming the DNA sequence into an IP address.

9. A computer-readable medium containing instructions that when executed by a processor, perform the method of claim 1.

10. A system for converting a DNA sequence associated with a user into an Internet Protocol (IP) address, the system comprising:
a processor;
memory; and
a DNA/IP converter, wherein the DNA-IP converter obtains a DNA sequence, transforms the DNA sequence into an Internet Protocol (IP) address, and accesses information associated with the IP address from a resource.

11. The system of claim 10, wherein the IP address is an Internet Protocol Version 6 (IPv6) address.

12. The system of claim 10, wherein the information associated with the IP address includes at least one of health information, location information, and financial information.

13. The system of claim 10, wherein the DNA sequence is unique for the user.

14. The system of claim 10, wherein the
obtaining a destination IP address for the resource; and
packaging the destination IP address and the IP address transformed from the DNA sequence into a communication packet.

15. The system of claim 14, further comprising a communication interface, wherein the DNA-IP converter transmits the communication packet over a communication network utilizing the communication interface to a location associated with the destination IP address.

16. The system of claim 10, wherein a second DNA-IP converter receives the IP address, converts the IP address into a DNA sequence, and synthesizes the DNA sequence into a nucleic acid molecule.

17. The system of claim 10, further comprising a DNA sampling interface, wherein the DNA sampling interface obtains a sample of DNA and sequences at least a portion of the DNA sample, and wherein the DNA-IP converter transforms the DNA sequence into an IP address.

18. A method for encoding an Internet Protocol (IP) address into a DNA sequence to digitally define a living organism, the method comprising:
obtaining an IP address;
transforming the IP address into a DNA sequence; and
synthesizing the DNA sequence, wherein the DNA sequence corresponds to the IP address.

19. The method of claim 18, wherein the IP address is an Internet Protocol Version 6 (IPv6) address.

20. The method of claim 19, wherein the IP address is associated with a user.


SEQUENCE LISTING (3 sequences)

SEQ ID NO: 1 - DNA, 18 bp, Artificial Sequence
  Synthetic
atcgattgagctctagcg

SEQ ID NO: 2 - DNA, 32 bp, Artificial Sequence
  Synthetic
agaaggggagacggatggaaggggagggagat

SEQ ID NO: 3 - DNA, 64 bp, Artificial Sequence
  Synthetic
agaaggggagacggatggaaggggagggagatgcacaatgggatcatggaaatcccatggacta